(19) United States
(12) Patent Application Publication
(10) Pub. No.: US 2016/0303364 A1
(43) Pub. Date: Oct. 20, 2016

(54) Medical Glove for Electric Stimulation

(71) Applicant: Larry Osborne, Albuquerque, NM (US)

(72) Inventor: Larry Osborne, Albuquerque, NM (US)

(21) Appl. No.: 15/132,066

(22) Filed: Apr. 18, 2016

Related U.S. Application Data
(60) Provisional application No. 62/149,413, filed on Apr. 17, 2015.

(51) Int. Cl. A61N 1/04; A61N 1/36

(52) U.S. Cl. CPC A61N 1/0484; A61N 1/0452; A61N 1/0456; A61N 1/36021

(57) ABSTRACT

A medical glove for electrical stimulation provides electrodes, supplying low voltage electric current for the purpose of Transcutaneous Electrical Nerve Stimulation (TENS) for patients with chronic pain in their hands, or Electrical Muscle Stimulation (EMS) to assist stroke patients in regaining motor skills. The glove is battery-powered, fits tightly on the user's hand, and is preferably manufactured from nylon fabric.

### CROSS-REFERENCE TO RELATED APPLICATIONS

This Application claims the benefit of U.S. Provisional Application No. 62/149,413, filed Apr. 17, 2015, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to medical devices and in particular to a medical glove for electric stimulation. People suffering from chronic pain in their hands, such as rheumatoid arthritis patients or stroke patients, face a broad array of challenges during their recovery. Most critical is regaining motor skills for full control of one or both of their hands. For both of these types of patients, such mundane daily tasks as fastening buttons, using a cellular telephone, tying a shoelace, or eating lunch may be difficult or impossible without the assistance of others. Fortunately, Transcutaneous Electrical Nerve Stimulation (TENS) and Electrical Muscle Stimulation (EMS) have proven to be effective in clinical studies as a therapeutic method for stimulating nerves and muscle contraction, respectively. A medical glove for electric stimulation, which stimulates muscles or nerves of the hand with battery-powered electrical pulses, would help to resolve these problems.

### SUMMARY OF THE INVENTION

Accordingly, the invention is directed to a medical glove for electrical stimulation. The glove is battery-powered, fits tightly on the user's hand, and is preferably manufactured from nylon fabric. The interior surface of the glove provides electrodes, supplying low voltage electric current for the purpose of Transcutaneous Electrical Nerve Stimulation (TENS) for patients with chronic pain in their hands, or Electrical Muscle Stimulation (EMS) to assist stroke patients in regaining motor skills.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

FIG. 1 is a front transparency view of the first exemplary embodiment, displaying the glove 10, the control unit 11, and the wires 12.

FIG. 2 is a rear view of the first exemplary embodiment, displaying the glove 10.

FIG. 3 is a side view of the first exemplary embodiment, displaying the glove 10, and the wires 12.

FIG. 4 is a rear sliced view of the first exemplary embodiment, displaying the interior of the glove 13, the electrodes 14, and the wires 12.

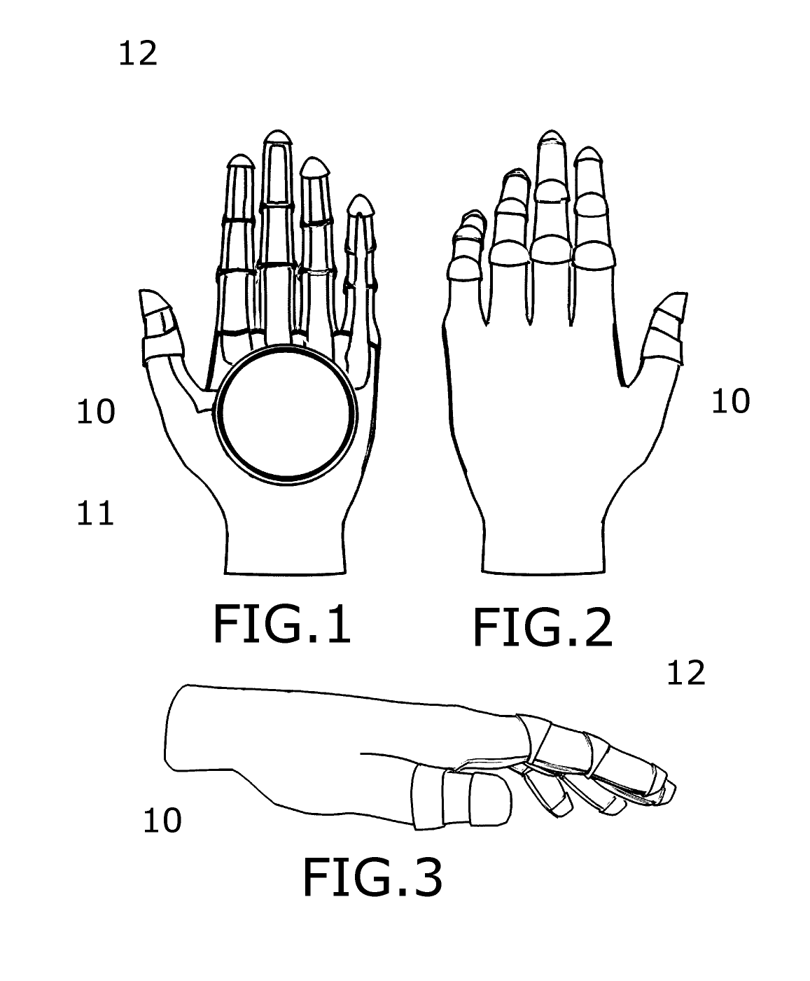

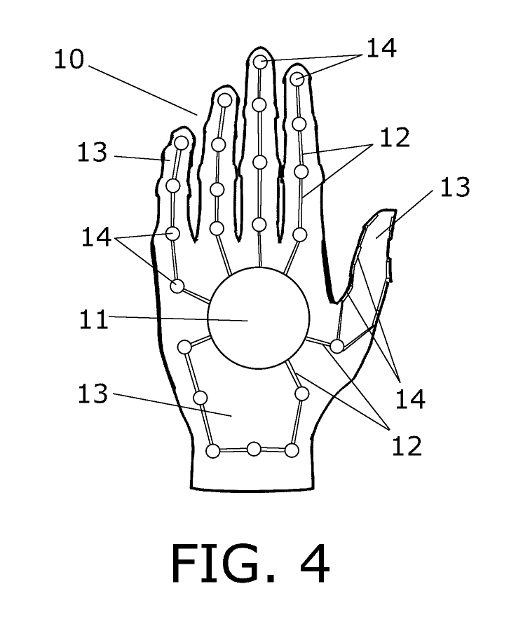

### DETAILED DESCRIPTION OF THE INVENTION

Referring now to the invention in more detail, the invention is directed to a medical glove 10 for electrical stimulation.

It is to be understood that while the invention is intended for use by patients with chronic hand pain and stroke patients, this is not intended as a limitation, but as a series of exemplary applications for the invention. With suitable modifications or with no modifications at all, the invention may be designed for and worn on parts of the body other than the hands, and used by other types of patients, such as stockings for patients with diabetic foot pain.

The first exemplary embodiment provides a glove 10 which is battery-powered, fits tightly on the user's hand, and preferably manufactured from nylon fabric. The interior surface 13 of the glove 10 provides electrodes 14, connected to a control unit 11 in the palm of the glove 10 via wires 12. The control unit 11 provides a replaceable battery and an on/off switch. The electrodes supply low voltage electric current for the purpose of Electrical Muscle Stimulation (EMS) to assist stroke patients in regaining motor skills.

To use the first exemplary embodiment, the user wears a glove 10 on each hand which is affected, and activates the on/off switch. The glove 10 is worn for a discrete therapeutic period as may be recommended by a physician, such as a period of 30 minutes per day.

The second exemplary embodiment is similar in structure and function to the first exemplary embodiment, with the following modifications. Instead of EMS, the electrodes supply electric current for the purpose of Transcutaneous Electrical Nerve Stimulation (TENS) for patients with chronic pain in their hands.

To use the second exemplary embodiment, the user wears a glove 10 on each hand which is affected, and activates the on/off switch. The glove 10 is worn for any length of time desired by the user for the relief of chronic pain in the hand.

The glove 10 is preferably manufactured from nylon fabric. The control unit 11 is preferably manufactured from a flexible, durable material, such as silicone or rubber, containing electrical components which are preferably manufactured from rigid, durable materials such as steel, aluminum alloy, plastic, copper alloy, and brass. The wires 12 are preferably manufactured from braided copper alloy wire sheathed in plastic. The electrodes, the battery, and the on/off switch are preferably manufactured from rigid, durable materials such as steel, aluminum alloy, plastic, and copper alloy.

Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A medical extremity cover, comprising: an extremity cover having an interior surface; a control unit; said control unit being attached to said extremity cover; a plurality of electrodes being attached to said interior surface; said plurality of electrodes being connected to said control unit via wires; said control unit providing a battery; said control unit providing low voltage electric current to said plurality of electrodes.

2. The medical extremity cover of claim 1, wherein said extremity cover is a glove having a palm, four fingers, and a thumb; said control unit being attached at the palm of said glove.

3. The medical extremity cover of claim 1, wherein said extremity cover is a stocking configured to fit a foot of a user.
